(19) United States
(12) Patent Application Publication
(10) Pub. No.: US 2024/0091416 A1
(43) Pub. Date: Mar. 21, 2024

(54) COMPOSITION FOR PRODUCING A BONE REPLACEMENT MATERIAL, METHOD FOR PRODUCING A PHARMACEUTICAL EXCIPIENT, PHARMACEUTICAL EXCIPIENT, AND USE THEREOF

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/460,146

(22) Filed: Sep. 1, 2023

(30) Foreign Application Priority Data
Sep. 8, 2022 (EP) 22194533.0

(51) Int. Cl. A61L 27/54; A61K 31/7036; A61K 31/7056; A61K 38/14; A61K 47/02; A61K 47/34; A61L 27/04; A61L 27/12; A61L 27/18

(52) U.S. Cl. CPC A61L 27/54; A61K 31/7036; A61K 31/7056; A61K 38/14; A61K 47/02; A61K 47/34; A61L 27/047; A61L 27/12; A61L 27/18; A61L 2300/232; A61L 2300/25; A61L 2300/404; A61L 2430/02

(57) ABSTRACT

A composition for producing a bone replacement material, which composition reacts with water or an aqueous solution in a cementitious setting reaction and forms a solid body. Additionally disclosed is a method for producing a pharmaceutical excipient having the aforementioned composition, the pharmaceutical excipient produced in this way, and the use of such a composition or such a pharmaceutical excipient for the local release of at least one pharmaceutical active ingredient.

### CROSS-REFERENCE TO RELATED APPLICATIONS

This application claims priority pursuant to 35 U.S.C. 119(a) to European Application No. 22194533.0, filed Sep. 8, 2022, which application is incorporated herein by reference in its entirety.

### FIELD

The invention relates to a composition for producing a bone replacement material, which composition reacts with water or an aqueous solution in a cementitious setting reaction and forms a solid body.

The invention further relates to a method for producing a pharmaceutical excipient having such a composition, the pharmaceutical excipient produced in this way, and the use of such a composition or such a pharmaceutical excipient for the local release of at least one pharmaceutical active ingredient.

### BACKGROUND

Compositions which react with water or an aqueous solution in a cementitious setting reaction to form a solid body-so-called mineral reactive systems such as cements and plasters—have long been used for a large number of applications. Typically, they consist of mineral powders which are mixed with water and achieve their final composition and therefore strength within a few minutes and up to even months. The setting reaction usually consists in dissolving water-soluble powder components and subsequently precipitating a more stable or heavy soluble phase or salt form, or in recrystallizing metastable powder components into a modification that is thermodynamically stable under the conditions of use. Frequently, two or more water-soluble powder components also react to form a sparingly soluble substance.

Often, parts of inorganic fillers are added to the compositions, which can influence the setting reaction and/or the strength of the formed solid body.

Such compositions have also been increasingly used for several years in medical applications such as dental fillings (e.g., root fillings) or also as bone replacement materials, such as, for example, in oro-maxillofacial surgery and orthopedics. Particularly in orthopedics, high demands are placed on the purity, biocompatibility, and also the setting behavior of the compositions, in particular their volume stability while setting and the speed of the setting reaction.

In particular, the use of calcium sulfate for filling bone cavities has been known since 1892 from the work of Dreesmann (H. Dreesman: Ueber Knochenplombierung. Beitr. Klin. Chir. 9 (1892) 804-810.). A large number of self-curing bone replacement materials based on calcium sulfate hemihydrate have been proposed. These are based on powdered calcium sulfate hemihydrate which is mixed with water or aqueous solutions, wherein a paste is produced which cures within a few minutes while forming calcium sulfate dihydrate. Furthermore, a large number of inorganic bone replacement materials are known with which metastable calcium phosphates cure after being mixed with water or aqueous solutions in the presence of suitable accelerators. Examples of such compositions for the production of bone replacement materials, which cure by mixing calcium sulfates and/or calcium phosphates with water or aqueous solutions, include the following patents: EP 1 301 219 B1, EP 1 601 387 B1, EP 1 948 255 B1, EP2 988 789 B1, EP3 157 862 B1, and EP3 166651B1.

EP 2 170 245 B1 describes a bone replacement material based on a hydraulic cement, wherein its particulate components are mixed with an organic carrier liquid while providing a paste, suspension, or dispersion. The carrier liquid has a water absorption of less than 25 by volume, i.e., is insoluble with water in the broadest sense, and forms emulsions with water. The composition preferably contains surfactants.

A disadvantage of such non-water-soluble carrier liquids is that the water or the aqueous solution, which is taken up in the composition for curing, leads to an increase in the original volume of the composition, since the carrier liquid is not completely replaced by the water or the aqueous solution. This increase in volume is disadvantageous in particular when filling cavities in bones, since it can lead to damage of the surrounding bone tissue. Furthermore, in such carrier liquids, pores are formed which adversely affect the strength of the bone replacement material.

Although the increase in volume can be reduced by the use of surfactants, the use of surfactants in the human body should be avoided due to their potentially membrane-damaging effect.

Furthermore, oligomeric or polymeric derivatives of ethylene glycol and propylene glycol, which are at least partially water-soluble, are proposed as admixtures to the non-water-soluble components of the carrier liquids.

WO 2002/062721 A1 and WO 2004/093734 A2 disclose composition based on calcium phosphates with a carrier liquid of glycerol, which can cure with water or aqueous solutions to form a solid body. Although glycerol circumvents the problem of an increase in volume of non-water-soluble carrier liquids during curing, the general disadvantages of glycerol-based carrier liquids, in particular, reduced storage stability, cannot be solved even by the disclosed addition of acids and gel formers.

### OBJECTS

It is an object of the present invention to at least partially overcome one or more of the disadvantages resulting from the prior art.

In particular, the invention is based on the aim of providing compositions which set to form solid bodies with water or aqueous solutions in a cementitious setting reaction and which, during setting, do not undergo any substantial increase in volume and should be stable in storage as long as possible. The compositions are intended to be used as bone replacement material. The compositions are intended to be made plastically in any desired form by a user. The compositions are intended to be introduced into cavities, and in particular bone cavities, without time pressure and to remain there due to their viscosity until they are set. Furthermore, the compositions are intended to be set from contact between the composition surface with water or aqueous solutions, such as for example body fluids, and in particular wound secretions. In this case, the compositions are intended to set without the need for mechanical mixing with water or aqueous solutions. The composition is therefore intended to function as a one-component system for producing a bone replacement material. The setting is intended to yield a dimensionally-stable solid body. The compositions shall consist of materials that are as toxicologically harmless as possible. As much as possible, the compositions are not intended to be membrane-damaging.

Another object of the invention is to provide a method for producing a pharmaceutical excipient by means of such a composition. The pharmaceutical excipients are intended to be used as bone replacement material for the local administration of a pharmaceutical active ingredient.

### Preferred Embodiments of the Invention

The features of the independent claims contribute to at least partially fulfilling at least one of the aforementioned objects. The dependent claims provide preferred embodiments which contribute to at least partially fulfilling at least one of the objects.

A first embodiment of the invention is a composition for producing a bone replacement material, which composition reacts with water or an aqueous solution in a cementitious setting reaction and forms a solid body consisting of:
a. at least one water-soluble polyether having a melting temperature of less than 25° C., wherein the at least one polyether has a water content of less than 1 wt %, relative to the total weight of the at least one polyether,
b. at least one particulate calcium-containing salt of the sulfuric acid and/or of the phosphoric acid, which reacts in a cementitious setting reaction upon contact with water or an aqueous solution, and which is not substantially soluble in the at least one polyether,
c. at least one particulate catalyst which accelerates the setting reaction of the composition with water, and which is not substantially soluble in the at least one polyether,
d. 0 to 10 wt. %, relative to the total weight of the composition, of at least one pharmaceutical active ingredient,
e. 0 to 30 wt. %, relative to the total weight of the composition, of at least one particulate inorganic filler, which is not substantially soluble in the at least one polyether.

In one embodiment, the composition is formed as a plastically deformable paste which, upon contact with water or an aqueous solution, cures, on its surface, substantially completely. This embodiment is a second embodiment of the invention, which is preferably dependent on the first embodiment of the invention.

In one embodiment of the composition, the at least one polyether has a proportion, and in particular a proportion by weight, of 20-40 wt. %, preferably of 20-35 wt. %, and more preferably of 20-30 wt. %, of the total weight of the composition. This embodiment is a third embodiment of the composition, which is preferably dependent on the first or the second embodiment of the invention.

In one embodiment of the composition, the at least one calcium-containing salt has a proportion, and in particular a proportion by weight, of 40-79 wt. %, preferably of 50-75 wt. %, and more preferably of 55-65 wt. %, of the total weight of the composition. This embodiment is a fourth embodiment of the invention, which is preferably dependent on one of the preceding embodiments of the invention.

In one embodiment of the composition, the at least one calcium-containing salt is calcium sulfate hemihydrate (CAS No. 10034-76-1), calcium sulfate anhydrite (CAS No. 7778-18-9), tricalcium phosphate (CAS No. 7758-87-4), tetracalcium phosphate (CAS No. 1306-01-0), or a mixture of the same, wherein calcium sulfate hemihydrate (CAS No. 10034-76-1) is preferred. This embodiment is a fifth embodiment of the invention, which is preferably dependent on one of the preceding embodiments of the invention.

In one embodiment of the composition, the at least one polyether is a polyethylene glycol, preferably polyethylene glycol 200, polyethylene glycol 400, and/or polyethylene glycol 600, and more preferably polyethylene glycol 400. This embodiment is a sixth embodiment of the invention, which is preferably dependent on one of the preceding embodiments of the invention.

In one embodiment of the composition, the at least one catalyst is calcium hydrogen phosphate, ammonium sulfate, and/or potassium sulfate, preferably ammonium sulfate and/or potassium sulfate, and more preferably ammonium sulfate or potassium sulfate. This embodiment is a seventh embodiment of the invention, which is preferably dependent on one of the preceding embodiments of the invention.

In one embodiment of the composition, the at least one filler is magnesium carbonate, hydroxylapatite, and/or calcium carbonate, preferably calcium carbonate. This embodiment is an eighth embodiment of the invention, which is preferably dependent on one of the preceding embodiments of the invention.

In one embodiment of the composition, the at least one pharmaceutical active ingredient is an antimicrobial active ingredient, preferably gentamicin, tobramycin, amikacin, vancomycin, teicoplanin, ramoplanin, dalbavancin, daptomycin, clindamycin and/or fosfomycin, and/or an antimycotic agent, preferably amphotericin B, fluconazole, ketoconazole, miconazole, and griseofulvin, caspofungin, micafungin, and/or anidulafungin. This embodiment is a ninth embodiment of the invention, which is preferably dependent on one of the preceding embodiments of the invention.

In one embodiment of the composition, the calcium-containing salt has a maximum particle size of 200 μm, preferably 100 μm, and more preferably 64 μm. This embodiment is a tenth embodiment of the invention, which is preferably dependent on one of the preceding embodiments of the invention.

In one embodiment, the composition is substantially free, preferably free, of surfactants. This embodiment is an eleventh embodiment of the invention, which is preferably dependent on one of the preceding embodiments of the invention.

A twelfth embodiment of the invention is a method for producing a pharmaceutical excipient, comprising the steps of:
a. providing a composition according to one of the first to eleventh embodiments of the invention,
b. providing a pharmaceutical active ingredient,
c. mechanically mixing the composition and the pharmaceutical active ingredient.

In one embodiment of the method, the pharmaceutical active ingredient is a particulate pharmaceutical active ingredient. This embodiment is a thirteenth embodiment of the invention, which is preferably dependent on the twelfth embodiment of the invention.

A fourteenth embodiment of the invention is a pharmaceutical excipient produced according to one of the twelfth or thirteenth embodiments of the invention.

A fifteenth embodiment of the invention is a use of a composition according to one of the first to twelfth embodiments of the invention or of a pharmaceutical excipient according to the fourteenth embodiment of the invention for local release of at least one pharmaceutical active ingredient.

### General

In the present description, range specifications also include the values specified as limits. An indication of the type “in the range of X to Y” with respect to a variable A consequently means that A can assume the values X, Y and values between X and Y. Ranges delimited on one side of the type “up to Y” for a variable A accordingly mean, as a value, Y and less than Y.

Some of the described features are linked to the term “substantially.” The term “substantially” is to be understood as meaning that, under real conditions and manufacturing techniques, a mathematically exact interpretation of terms such as “superimposition,” “perpendicular,” “diameter,” or “parallelism” can never be given exactly, but only within certain manufacturing-related error tolerances. For example, “substantially perpendicular axes” form an angle of 85 degrees to 95 degrees relative to one another, and “substantially equal volumes” comprise a deviation of up to 5 vol %. A “device consisting substantially of plastic material” comprises, for example, a plastics content of ≥95 to ≤100 wt. %. “Adding substantially all of a volume B” comprises, for example, adding ≥95 to ≤100 vol % of the total volume of B. A “substantially fully cured composition” is cured, for example, at ≥95 to ≤100 wt. %, relative to the total weight of the composition, wherein the components not yet cured remain either uncured or subsequently cure over time. For example, the substance X is not actively added to a “composition substantially free of substance X.” A “substance Y which is substantially insoluble in a liquid X” is understood to mean a substance Y which, under normal conditions (25° C. and 101.3 kPa), does not dissolve more than up to 0.1 mol/L in the liquid X.

### DETAILED DESCRIPTION

A first subject matter of the invention relates to a composition for producing a bone replacement material, which composition reacts with water or an aqueous solution in a cementitious setting reaction and forms a solid body consisting of:
a. at least one water-soluble polyether having a melting temperature of less than 25° C., and in particular having a melting temperature of less than 25° C. at a pressure of 101.3 kPa, wherein the at least one polyether has a water content of less than 1 wt. %, relative to the total weight of the at least one polyether,
b. at least one particulate calcium-containing salt of the sulfuric acid and/or of the phosphoric acid, which reacts in a cementitious setting reaction upon contact with water or an aqueous solution, and which is not substantially soluble in the at least one polyether,
c. at least one particulate catalyst which accelerates the setting reaction of the composition with water, and which is not substantially soluble in the at least one polyether,
d. 0 to 10 wt. %, relative to the total weight of the composition, of at least one pharmaceutical active ingredient,
e. 0 to 30 wt. %, relative to the total weight of the composition, of at least one particulate inorganic filler, which is not substantially soluble in the at least one polyether.

The invention relates to a composition. According to the invention, a composition is understood to be a mixture, and preferably a pasty mixture, consisting of the components a., b., c., and optionally d. and/or e. as claimed, which reacts with water or an aqueous solution in a cementitious setting reaction and forms a solid. The composition is therefore a hydraulically-curable composition. Preferably, the consistency of the composition under normal conditions (25° C. and 101.3 kPa) does not exceed that of a kneadable paste, and in particular a manually kneadable paste. The solid which can be obtained from the composition by the cementitious setting reaction can be used as a bone replacement material.

The components of the composition add up to 100 wt. %, relative to the total weight of the composition. In other words, the composition can consist of the components a., b., and c., the components a., b., c., and d., the components a., b., c., and e., or the components a., b., c., d., and e.

With the component b., the composition comprises reactive, particulate, calcium-containing salts of sulfuric acid, phosphoric acid, or sulfuric acid and phosphoric acid, which react with water or aqueous solutions in a cementitious setting reaction and therefore form a solid. During the cementitious setting reaction, the components b. of the composition come into contact with water or an aqueous solution so that these components at least partially dissolve and recrystallize in water, or can precipitate again in the aqueous phase and therefore form an, in particular, coherent, solid body which can function as a bone replacement material.

According to the invention, one or more calcium ions are used as cations of the salts of the sulfuric acid and/or of the phosphoric acid. Calcium has the advantage that it is physiologically harmless, in contrast to other cations such as, for example, magnesium. A calcium-containing salt is understood here to mean a salt which mainly contains calcium as a cation or cations. Also included are those salts which contain additional cations, and in particular Na, K, NH₄, or Mg, possibly also as impurities, wherein pure calcium-containing salts are preferred.

As component c., the composition contains at least one particulate catalyst which accelerates the setting reaction of the composition with water. The at least one catalyst is selected on the basis of component b. of the composition, since different Ca-containing salts of the sulfuric acid and/or of the phosphoric acid cure with certain catalysts at different speeds in the cementitious setting reaction.

With component d. as an optional component, the composition contains a pharmaceutical active ingredient which, after setting from the cured composition, can be delivered to the applied site in the patient. Suitable pharmaceutical active ingredients are all substances that are relevant for the local treatment of bone diseases and the adjacent tissues. The pharmaceutical active ingredients can support the function of the bone replacement material in the desired manner. In this case, particulate, e.g., flake-like and/or lyophilized, and/or liquid pharmaceutical active ingredients can be used.

With component e. as another optional component, the composition comprises at least one particulate inorganic filler. A filler is in particular integrated into the cement which forms during setting, but is basically only indirectly involved in the cementitious setting reaction. Participation can consist, for example, in the fact that a filler functions as a crystallization nucleus for mineralization during the cementitious setting reaction and therefore in particular influences the setting kinetics, but is itself not substantially converted.

In order to combine the components b., c., and optionally d. and/or e. into a moldable composition, preferably kneadable under normal conditions, the composition contains a water-soluble polyether which has a melting temperature of less than 25° C., i.e., a liquid polyether under normal conditions, wherein the at least one polyether has a water content of less than 1 wt. %, relative to the total weight of the at least one polyether. The at least one polyether wets the particulate components of the composition at least partially, and preferably substantially completely, so that a shapeable, and preferably homogeneous, paste arises which can be introduced into a cavity, in particular, into a cavity of a bone, where it remains on account of its viscosity until it sets with water or an aqueous solution, in particular a body fluid such as wound secretion, in the cavity in a cementitious setting reaction to form a solid body and therefore acts as a bone substitute material.

A polyether is understood to mean substances which comprise at least four ether groups in the individual molecule. Preferably, the polyethers are linear molecules with at least four ether groups.

It is essential that the polyether has a water content of less than 1 wt. %, and preferably less than 0.5 wt. %, relative to the total weight of the at least one polyether, so that the composition will be stable in storage until it is applied to its desired application site for setting. Preferably, the polyether is substantially free of water.

In addition to any optional liquid pharmaceutical active ingredients and/or any impurities, the composition therefore does not contain any (additional) liquids, and in particular no water-soluble liquids, beyond the at least one polyether. The polyethers according to the invention are soluble in water, preferably in any mixture ratios. This allows a rapid penetration of water or aqueous solutions into the composition, or an at least partial, and preferably substantially complete, exchange of the polyether of the composition with water or an aqueous solution after application of the composition in a patient, as a result of which the cementitious setting reaction occurs with the formation of a solid body. This solid body serves as a bone replacement material. The at least one polyether allows substantially complete setting of the composition, even if it is brought into contact with water or an aqueous solution, i.e., with body fluids of a patient, only on its surface, or at least part of its surface. An in particular mechanical mixing of the composition with water or an aqueous solution is unnecessary for setting the composition substantially completely. Furthermore, even when its components merely superficially contact water or an aqueous solution, the composition can set to become a solid, in particular by the exclusive use of the at least one polyether, within a few minutes, e.g., within 5 minutes, at least on its surface, in particular up to a layer thickness of more than 1 mm. The exact time until the substantially complete formation of a solid body depends as expected on the amount of composition to set, its given shape, in particular its ratio of surface to mass, and its exposure to water or an aqueous solution.

Surprisingly, despite the, in particular exclusive, use of the at least one polyether as the water-soluble liquid, the compositions are stable in storage over a longer period, e.g., over several months, such as for example more than three months, until they react in a cementitious setting reaction when they come into contact with water or an aqueous solution. The compositions are, for example, therefore stable in storage if the original viscosity of the compositions remains substantially unchanged, and/or the compositions over the storage period remain a pliable, kneadable, and moldable paste. Other water-soluble liquids as the excipient in such compositions, e.g., glycerol, however, have not proven to be stable in storage as long. In order to provide the composition as a moldable paste which can be reliably introduced into cavities of a patient by virtue of its viscosity and which reacts with water or with an aqueous liquid under a cementitious setting reaction, the particulate components of the composition, in particular, the components b., c., and, if present, e., are not substantially soluble in the at least one polyether.

The composition can be present in different viscosities before the cementitious setting reaction, as determined by the proportion by weight of the composition, in particular the ratio of particulate to liquid components of the composition.

One embodiment of the composition is characterized in that the composition is formed as a plastically deformable paste which, upon contact with water or an aqueous solution, cures, on its surface, substantially completely. In comparison to a paste with a lower viscosity, a plastically deformable paste has the advantage that it can be introduced into a cavity in such a way that the composition can be removed from this cavity solely due to gravity. A plastically deformable paste furthermore has a viscosity which permits manual deformation. The composition can therefore also be changed into any shape without tools, such as a knife for cutting or carving. This makes it easier for a surgeon to use the composition during an operation.

One embodiment of the composition is characterized in that the at least one polyether has a proportion, and in particular a proportion by weight, of 20-40 wt. %, and preferably of 20-30 wt. %, of the total weight of the composition. This allows substantially homogeneous mixing of the components of the composition, so that basically all particulate components are at least partially, and preferably basically completely, encased by the at least one polyether, which enables the most complete cementitious setting possible when the composition contacts, even only superficially contacts, water or an aqueous solution. Furthermore, the composition thereby has an extremely shapeable, and preferably pasty, viscosity which makes it easy to apply the composition in a patient.

One embodiment of the composition is characterized in that the at least one calcium-containing salt has a proportion, and in particular a proportion by weight, of 40-79 wt. % of the total weight of the composition. After the cementitious setting reaction, such compositions yield a stable solid body which can function well as a bone replacement material.

Different salts or mixtures thereof can be used as the at least one calcium-containing salt of the sulfuric acid and/or of the phosphoric acid, as long as these can enter into a cementitious setting reaction with water or an aqueous solution to form a solid body.

One embodiment of the composition is characterized in that the at least one calcium-containing salt is calcium sulfate hemihydrate, calcium sulfate anhydrite, tricalcium phosphate, tetracalcium phosphate, or any mixtures of the aforementioned salts, wherein calcium sulfate hemihydrate is preferred.

As the at least one polyether, it is possible to use any water-soluble polyethers, and preferably soluble in any mixture ratios with water, in pure form or in any mixtures, as long as they have a melting temperature of less than 25° C., and in particular a melting temperature of less than 25° C. at 101.3 kPa. For example, polymers of propylene glycol, i.e. polypropylene glycols, can be used. For example, polypropylene glycol 200 up to polypropylene glycol 600 can be used.

One embodiment of the composition is characterized in that the at least one polyether is a polyethylene glycol, a mixture of different polyethylene glycols, or a mixture of a polyethylene glycol with a polypropylene glycol, wherein a pure polyethylene glycol is preferred. Polyethylene glycols are preferred, since they are favorable, easily accessible, and in particular biologically highly compatible, water-soluble polyethers. Particularly preferred polyethylene glycols are polyethylene glycol 200, polyethylene glycol 400 and polyethylene glycol 600, wherein, of these, polyethylene glycol 400 is the most preferred. Polyethylene glycol 400 is preferred, since this yields a composition that is easy to handle due to its viscosity.

Various substances can be added as catalysts in pure form or in mixtures to accelerate the cementitious setting reaction of the composition.

One embodiment of the composition is characterized in that the at least one catalyst is calcium dihydrogen phosphate (CAS-7758-23-8), ammonium sulfate (CAS-7783-20-2), potassium sulfate (CAS No. 7778-80-5), or a mixture of two or more of the aforementioned substances. The at least one catalyst is preferably selected on the basis of the employed Ca-containing salts.

The at least one catalyst may be added to the composition in different proportions by weight to accelerate the cementitious setting reaction.

One embodiment of the composition is characterized in that the catalyst has a proportion, and in particular a proportion by weight, of 1-12 wt. % of the total weight of the composition. Proportions lower than 1 wt. % of catalyst slow down the cementitious setting reaction, so that, under certain circumstances, surgery becomes unnecessarily long, and there is a risk that the composition applied in the patient is removed from the desired location prior to the formation of the solid body or assumes an undesired shape. Although higher proportions of catalyst accelerate the formation of the solid, the catalyst contributes to the strength of the solid body to only a limited extent, so that the solid body can lose strength given higher proportions of the catalyst than 12 wt. %. Moreover, an even faster cementitious setting reaction is usually not relevant in practice.

Different substances in pure form or in mixtures can be added to the composition as particulate inorganic fillers.

One embodiment of the composition is characterized in that the at least one filler is magnesium carbonate, hydroxylapatite, calcium carbonate, or any mixture of two or more of the aforementioned substances, wherein calcium carbonate is preferred. Calcium carbonate is preferred, since it is particularly biocompatible.

As the optional at least one active pharmaceutical active ingredient, different substances can be added, in pure form or in mixtures of the composition, which are suitable for a local treatment of bone diseases and/or the adjacent tissues.

One embodiment of the composition is characterized in that the at least one pharmaceutical active ingredient is an antimicrobial active ingredient, an antimycotic agent, or a mixture of at least one antimicrobial and at least one antimycotic agent. Gentamicin, tobramycin, amikacin, vancomycin, teicoplanin, ramoplanin, dalbavancin, daptomycin, clindamycin, and fosfomycin are preferred as anti-microbial active ingredients, and amphotericin B, fluconazole, ketoconazole, miconazole, griseofulvin, caspofungin, micafungin, and anidulafungin are preferable as antimycotic agents.

The at least one calcium-containing salt of the sulfuric acid and/or of the phosphoric acid can have different particle sizes.

One embodiment of the composition is characterized in that the at least one calcium-containing salt has a maximum particle size of 200 μm, preferably at most 100 μm, and more preferably at most 64 μm. The maximum particle size can be obtained, for example, by selecting corresponding screening fractions of the respective calcium-containing salts.

Such particle sizes ensure as homogeneous a mixture as possible of the composition, and enable the fastest possible and uniform and formation of a solid by the cementitious setting reaction of the composition with water or an aqueous solution.

One embodiment of the composition is characterized in that the composition is substantially free of surfactants. Such compositions consist only of such ingredients a., b., c., and optionally d. and/or e., which have substantially no surfactant properties. Composition without surfactants are preferred, since surfactants have a membrane damaging effect and can therefore show negative effects on the patients to be treated with the composition. The term “surfactant” comprises in particular amphiphilic molecules which can also act as emulsifiers or wetting agents.

A further subject of the invention relates to a method for producing a pharmaceutical excipient comprising the steps of:
a. providing a composition according to one of the preceding embodiments,
b. providing an pharmaceutical active ingredient,
c. mechanically mixing the composition and the pharmaceutical active ingredient.

The composition provided in step a. of the method can already contain, as component d., one or more pharmaceutical active ingredients, or can be free of pharmaceutical active ingredients.

The pharmaceutical active ingredient provided in step b. of the method is preferably one or more of the pharmaceutical active ingredients already disclosed for the composition.

If the composition provided in step a. already contains a pharmaceutical active ingredient, or a mixture of pharmaceutical active ingredients, as component d., the pharmaceutical active ingredient provided in step b. of the method can also be the same active ingredient or the same mixture of pharmaceutical active ingredients. In this case, the method serves to increase the concentration of the corresponding pharmaceutical active ingredient or the corresponding mixture of pharmaceutical active ingredients in the provided composition.

In step c. of the method, the composition provided in step a. and the pharmaceutical active ingredient provided in step b. are mechanically mixed to provide a pharmaceutical excipient.

The mechanical mixing can be accomplished in different ways. For example, this can be carried out using an aid, such as a spatula or other suitable mixing tool. Preferably, the mechanical mixing takes place manually and therefore without aids, since this can be carried out quickly and easily for a surgeon, even during an operation. Manual mixing can also be termed kneading.

The pharmaceutical active ingredient provided in step b. of the method can be liquid.

One embodiment of the method is characterized in that the pharmaceutical active ingredient is a particulate active ingredient. For example, the pharmaceutical active ingredient can be powdered or flaked. In one embodiment, the pharmaceutical active ingredient is lyophilized. Particulate pharmaceutical active ingredients are easier for a surgeon in an ongoing operation to handle than liquid pharmaceutical active ingredients, in particular if the mechanical mixing in step c. of the method is carried out manually.

A further subject matter of the invention relates to a pharmaceutical excipient produced by such a method.

A composition containing a pharmaceutical active ingredient and a pharmaceutical excipient produced by the method may, apart from obvious differences in their components, differ in certain circumstances by the fact that the pharmaceutical active ingredient admixed to the composition by the method is distributed less homogeneously in the composition than if it were already present in the composition before the method. If the pharmaceutical active ingredient were already added during the production of the composition, it could be premixed with the particulate components of the composition before the composition is converted into a moldable composition by adding the at least one polyether. A “subsequent” mixing of the pharmaceutical active ingredient in this moldable paste can be made more difficult, depending on the viscosity of the composition. An advantage of the pharmaceutical excipient is that it can be intentionally adapted and provided for the needs of a patient directly in the operating room.

A further subject matter of the invention relates to a use of a composition according to one of the aforementioned embodiments, or of a pharmaceutical excipient according to one of the aforementioned embodiments, for local release of at least one pharmaceutical active ingredient, and in particular for the treatment of bone diseases and the tissues adjacent to bone.

An advantage of using the pharmaceutical excipient is its flexible inclusion of pharmaceutical active ingredients, even when it is produced in an ongoing operation. A surgeon is therefore able to quickly and easily add a, possibly additional, pharmaceutical active ingredient to the composition. Possible inhomogeneities are less important than this advantage.

The features disclosed for the composition are also disclosed for the method and the pharmaceutical excipient, and vice versa.

### Examples

In the following, by way of example, the invention is illustrated further by examples. The invention is not limited to the examples.

The following materials for the production of the examples of the compositions for producing a bone replacement material were used:
CSH: Calcium sulfate hemihydrate (CAS No. 10034-76-1)
CSAH: Calcium sulfate (anhydrite) (CAS No. 7778-18-9)
AS: Ammonium sulfate (CAS-7783-20-2)
CS: Potassium sulfate (CAS No. 7778-80-5)
CA: Calcium carbonate (CAS No. 471-34-1)
HA: Hydroxyapatite (CAS No. 1306-06-5)
G: Gentamicin sulfate
VHCl: Vancomycin hydrochloride
CL: Clindamycin hydrochloride
TCP: Tricalcium phosphate (CAS No. 7758-87-4)
CDHP: Calcium dihydrogen phosphate (CAS-7758-23-8)
TTCP: Tetralcalcium phosphate (CAS No. 1306-01-0)

The bone replacement materials were produced by first grinding the particulate components of the corresponding compositions by using porcelain balls as grinding media with the aid of a Turbula mixer. Subsequently, the particulate mixture obtained in this way was mixed with the respective liquids (polyethylene glycol 200 (PEG200) or polyethylene glycol 400 (PEG400) for exemplary embodiments 1 to 46 according to the invention or glycerol acetate, oleic acid, or Miglyol® 812 for comparative examples 1 to 10) to obtain the compositions in the form of viscous pastes. From the compositions obtained in this manner, balls with a diameter of about 10 mm were then manually formed, which were incorporated into distilled water. The progression of the cementitious setting reaction of the compositions in water was monitored by checking the strength of the balls. For this purpose, the strength of the balls was tested every minute by piercing with a spatula.

The compositions of the exemplary embodiments without addition of a pharmaceutical active ingredient can be stored for at least 3 months, essentially without changing the original viscosities and without curing.

The balls of exemplary embodiments 1 to 46 were all cured after about 5 minutes, at least on their surface. The cementitious setting reaction in the interior of the balls was then carried out within another few minutes. Furthermore, the balls formed from the compositions substantially maintained their shape and size while curing. The compositions therefore cure substantially without a significant change in volume.

With the balls of the comparative examples, an increase in volume within a range of approximately 5 to 15 by volume, relative to the original volume of the balls, occurred over the course of curing. Furthermore, the surfaces of the balls cracked.

| Exemplary | | | | | | |
|---|---|---|---|---|---|---|
| embodiment | CSH [g] | AS [g] | PEG200 | GS [g] | VHCL [g\|] | CL [g] |
| 1 | 8.5 | 1.5 | 3.0 | — | — | — |
| 2 | 9.0 | 1.0 | 3.0 | — | — | — |
| 3 | 9.5 | 0.5 | 3.0 | — | — | — |
| 4 | 9.5 | 0.5 | 3.0 | 0.5 | — | — |
| 5 | 9.5 | 0.5 | 3.0 | 0.3 | — | — |
| 6 | 9.5 | 0.5 | 3.0 | 0.1 | — | — |
| 7 | 9.5 | 0.5 | 3.0 | — | 0.5 | — |
| 8 | 9.5 | 0.5 | 3.0 | — | 0.3 | — |
| 9 | 9.5 | 0.5 | 3.0 | — | 0.1 | — |
| 10 | 9.5 | 0.5 | 3.0 | — | — | 0.5 |
| 11 | 9.5 | 0.5 | 3.0 | — | — | 0.3 |
| 12 | 9.5 | 0.5 | 3.0 | — | — | 0.1 |
| Exemplary | | | | | | |

| embodiment | CSH [g] | AS [g] | PEG400 | GS [g] | VHCL [g\|] | CL [g] |
|---|---|---|---|---|---|---|
| 13 | 8.5 | 1.5 | 3.0 | — | — | — |
| 14 | 9.0 | 1.0 | 3.0 | — | — | — |
| 15 | 9.5 | 0.5 | 3.0 | — | — | — |
| 16 | 9.5 | 0.5 | 3.0 | 0.5 | — | — |
| 17 | 9.5 | 0.5 | 3.0 | 0.3 | — | — |
| 18 | 9.5 | 0.5 | 3.0 | 0.1 | — | — |
| 19 | 9.5 | 0.5 | 3.0 | — | 0.5 | — |
| 20 | 9.5 | 0.5 | 3.0 | — | 0.3 | — |
| 21 | 9.5 | 0.5 | 3.0 | — | 0.1 | — |
| 22 | 9.5 | 0.5 | 3.0 | — | — | 0.5 |
| 23 | 9.5 | 0.5 | 3.0 | — | — | 0.3 |
| 24 | 9.5 | 0.5 | 3.0 | — | — | 0.1 |

| Exemplary | CSAH | | | | | | |
|---|---|---|---|---|---|---|---|
| embodiment | [g] | AS [g] | PEG200 | CA [g] | TCP [g\|] | HA [g] | GS [g] |
| 25 | 6.0 | 0.5 | 3.0 | 3.5 | — | — | 0.5 |
| 26 | 6.0 | 0.5 | 3.0 | — | 3.5 | — | 0.5 |
| 27 | 6.0 | 0.5 | 3.0 | — | — | 3.5 | 0.5 |

Exemplary

| embodiment | CSH [g] | KS [g] | PEG200 | GS [g] | VHCL [g\|] | CL [g] |
|---|---|---|---|---|---|---|
| 28 | 8.5 | 1.5 | 3.0 | — | — | — |
| 29 | 9.0 | 1.0 | 3.0 | — | — | — |
| 30 | 9.5 | 0.5 | 3.0 | — | — | — |
| 31 | 9.5 | 0.5 | 3.0 | 0.5 | — | — |
| 32 | 9.5 | 0.5 | 3.0 | 0.3 | — | — |
| 33 | 9.5 | 0.5 | 3.0 | 0.1 | — | — |
| 34 | 9.5 | 0.5 | 3.0 | — | 0.5 | — |
| 35 | 9.5 | 0.5 | 3.0 | — | 0.3 | — |
| 36 | 9.5 | 0.5 | 3.0 | — | 0.1 | — |
| 37 | 9.5 | 0.5 | 3.0 | — | — | 0.5 |
| 38 | 9.5 | 0.5 | 3.0 | — | — | 0.3 |
| 39 | 9.5 | 0.5 | 3.0 | — | — | 0.1 |
| Exemplary | TCP | TTCP | CDHP | PEG200 | GS | VHCl |
| embodiment | [g] | [g] | [g] | [g] | [g] | [g] |
| 40 | 6.00 | — | 4.00 | 4.00 | — | — |
| 41 | 6.00 | — | 4.00 | 4.00 | — | — |
| 42 | 6.00 | — | 4.00 | 4.00 | 0.74 | — |
| 43 | 6.00 | — | 4.00 | 4.00 | — | 0.74 |
| 44 | — | 5.00 | 5.00 | 4.00 | — | — |
| 45 | — | 5.50 | 4.50 | 4.00 | — | — |
| 46 | — | 5.50 | 4.50 | 4.00 | 0.74 | — |

| Comparative | | | Miglyol ® | |
|---|---|---|---|---|
| example | CSH [g] | AS [g] | 812 [g] | GS [g] |
| 1 | 9.5 | 0.5 | 3.0 | 0.5 |
| 2 | 9.5 | 0.5 | 3.0 | 0.3 |
| 3 | 9.5 | 0.5 | 3.0 | 0.1 |
| Comparative | | | Glycerol | |
| example | CSH [g] | AS [g] | triacetate [g] | GS [g] |
| 4 | 9.5 | 0.5 | 3.0 | 0.5 |
| 5 | 9.5 | 0.5 | 3.0 | 0.3 |
| 6 | 9.5 | 0.5 | 3.0 | 0.1 |
| Comparative | | | Oleic Acid | |

| example | CSH [g] | AS [g] | [g] | GS [g] |
|---|---|---|---|---|
| 7 | 9.5 | 0.5 | 3.0 | 0.5 |
| 8 | 9.5 | 0.5 | 3.0 | 0.3 |
| Comparative | TCP | CDHP | Miglyol ® | GS |
| example | [g] | [g] | 812 [g] | [g] |
| 10 | 6.00 | 4.0 | 4.00 | 0.74 |

What is claimed is:

1. A composition for producing a bone replacement material, which composition reacts with water or an aqueous solution in a cementitious setting reaction and forms a solid body consisting of:
a. at least one water-soluble polyether having a melting temperature of less than 25° C., wherein the at least one polyether has a water content of less than 1 wt. %, relative to the total weight of the at least one polyether,
b. at least one particulate calcium-containing salt of the sulfuric acid and/or of the phosphoric acid, which reacts in a cementitious setting reaction upon contact with water or an aqueous solution, and which is not substantially soluble in the at least one polyether,
c. at least one particulate catalyst which accelerates the setting reaction of the composition with water, and which is not substantially soluble in the at least one polyether,
d. 0 to 10 wt. %, relative to the total weight of the composition, of at least one pharmaceutical active ingredient,
e. 0 to 30 wt. %, relative to the total weight of the composition, of at least one particulate inorganic filler, which is not substantially soluble in the at least one polyether.

2. The composition according to claim 1, wherein the composition is formed as a plastically deformable paste which, upon contact with water or an aqueous solution, cures, on its surface, substantially completely.

3. The composition according to claim 1, wherein the at least one polyether has a proportion of 20-40 wt. % of the total weight of the composition.

4. The composition according to claim 1, wherein the at least one calcium-containing salt has a proportion of 40-79 wt. % of the total weight of the composition.

5. The composition according to claim 1, wherein the at least one calcium-containing salt is calcium sulfate hemihydrate, calcium sulfate anhydrite, tricalcium phosphate, and/or tetracalcium phosphate.

6. The composition according to claim 1, wherein the at least one polyether is a polyethylene glycol, preferably polyethylene glycol 200, polyethylene glycol 400, and/or polyethylene glycol 600.

7. The composition according to claim 1, wherein the at least one catalyst is calcium dihydrogen phosphate, ammonium sulfate, and/or potassium sulfate.

8. The composition according to claim 1, wherein the at least one filler is magnesium carbonate, hydroxyapatite, and/or calcium carbonate.

9. The composition according to claim 1, wherein the at least one pharmaceutical active ingredient is an antimicrobial active ingredient, preferably gentamicin, tobramycin, amikacin, vancomycin, teicoplanin, ramoplanin, dalbavancin, daptomycin, clindamycin and/or fosfomycin, and/or an antimycotic agent, preferably amphotericin B, fluconazole, ketoconazole, miconazole, and griseofulvin, caspofungin, micafungin, and/or anidulafungin.

10. The composition according to claim 1, wherein the at least one calcium-containing salt has a maximum particle size of 200 μm, preferably 100 μm, and more preferably 64 μm.

11. The composition according to claim 1, wherein the composition is substantially free of surfactants.

12. A method for producing a pharmaceutical excipient, comprising the steps of:
a. providing a composition according to claim 1,
b. providing a pharmaceutical active ingredient,
c. mechanically mixing the composition and the pharmaceutical active ingredient.

13. The method according to claim 12, wherein the pharmaceutical active ingredient is a particulate pharmaceutical active ingredient.

14. A pharmaceutical excipient produced according to claim 12.

15. The use of a composition according to claim 1 for local release of at least one pharmaceutical active ingredient.
